(19) United States
(12) Patent Application Publication
(10) Pub. No.: US 2023/0323291 A1
(43) Pub. Date: Oct. 12, 2023

(54) MINI-BRAIN STRUCTURE AND CONSTRUCTION METHOD THEREFOR

(71) Applicants: SOGANG UNIVERSITY RESEARCH & BUSINESS DEVELOPMENT FOUNDATION, Seoul (KR); UNIANCE GENE, Seoul (KR)

(72) Inventors: Jeong Woo CHOI, Seoul (KR); Soo Jeong PARK, Seoul (KR); Sang Nam LEE, Seoul (KR)

(21) Appl. No.: 18/028,156

(22) PCT Filed: Oct. 15, 2021
(86) PCT No.: PCT/KR2021/014414

§ 371 Date: Mar. 23, 2023

(30) Foreign Application Priority Data
Oct. 16, 2020 (KR) 10-2020-0134618

(51) Int. Cl. C12N 5/079

(52) U.S. Cl. CPC C12N 5/0618; C12N 2506/45

(57) ABSTRACT

The present invention relates to a mini-brain structure and a construction method therefor and, more specifically, to a mini-brain and a construction method therefor, wherein induced pluripotent stem cells can be used to prepare brain organoids for different brain regions accounting for the cerebrum, the mesencephalon, the cerebellum, the thalamus, and the like and the organoids are combined into single structures, thereby making it possible to further effectively implement actual brain functions and wherein brain organoids for different brain regions can be selectively combined into single structures according to purposes, thereby achieving the aim of using brain organoids and enhancing convenience and economical benefit.

### TECHNICAL FIELD

The present invention relates to a mini-brain structure and a method of forming the mini-brain structure. More particularly, the present invention relates to a mini-brain structure and a method of forming the mini-brain structure in which organoids present in respectively different regions in the brain, such as the cerebrum, midbrain, cerebellum, and thalamus, are formed by using induced pluripotent stem cells and are combined into a single organism, thereby further effectively enabling the realization of actual brain functions. In addition, depending on the purpose of use, the organoids presented in respectively different regions in the brain can be selectively combined into a single organism, thereby achieving the purpose of using the brain organoids while improving convenience and cost-effectiveness.

### BACKGROUND ART

Organoids are organ-like structures formed by reaggregating and recombining stem cells or cells isolated from organs through 3D culturing. Structures and functions of organs can be reproduced with the use of organoids, so research has been conducted widely on the application of organoids to new drug development and disease treatment.

Among the organoids, brain organoids have recently received attention. Brain organoids contain specific brain cells and can reproduce specific brain functions. As described in the patent document below, the brain organoids are formed through self-organization, self-proliferation, and tissue-specific lineage differentiation after culturing induced pluripotent stem cells in a medium containing a signal transducer in an in vivo-like substrate environment. Currently, a method of forming brain organoids, similar to specific brain regions, such as the midbrain, cerebellum, thalamus, hypothalamus, and the like, is widely known.

### PATENT DOCUMENT

Korean Patent Application Publication No. 10-2020-0081294 (published on 7 Jul. 2020) “METHOD OF FORMING BRAIN ORGANOID”

However, it is problematic to realize complex human brain functions, which are organically combined, only with existing specific brain organoids based on limited regions.

### DISCLOSURE

### Technical Problem

The present invention has been made to solve the above problems.

An objective of the present invention is to provide a mini-brain and a method of forming the mini-brain, in which organoids present in respectively different regions in the brain, such as the cerebrum, midbrain, cerebellum, and thalamus, are formed by using induced pluripotent stem cells and combined into a single organism, thereby further effectively enabling the realization of actual brain functions.

In addition, another objective of the present invention is to provide a mini-brain and a method of manufacturing the mini-brain, in which organoids presented in respectively different regions in the brain are selectively combined into a single organism, thereby achieving the purpose of using the brain organoids while improving convenience and cost-effectiveness.

### Technical Solution

The present invention is implemented by an embodiment having the following construction to achieve the above objectives.

According to one embodiment of the present invention, a mini-brain structure, according to the present invention, includes a plurality of brain organoids, in which the brain organoids of the plurality of brain organoids are present in respectively different regions in the brain, and the plurality of brain organoids is combined into a single organism.

According to another embodiment of the present invention, the mini-brain structure, according to the present invention, may be formed by selecting some organoids from the plurality of brain organoids and combining the selected organoids depending on a purpose of using the mini-brain structure.

According to a further embodiment of the present invention, the mini-brain structure, according to the present invention, may have a shape that varies depending on a combining position of the according to a purpose of using the mini-brain structure.

According to a further embodiment of the present invention, a method of forming a mini-brain structure, according to the present invention, includes: selecting organoids present in one or more specific brain regions depending on a purpose of using a mini-brain structure; regulating a culture medium composition by homogenizing each of the culture media in which the respective selected organoids are suspended; and mixing the homogenized culture media in one container to produce a mixed culture medium after the regulating of the culture medium composition so that the organoids become close to each other.

According to a further embodiment of the present invention, the method of forming the mini-brain structure, according to the present invention, may further include inducing each of the organoids to be combined into a single organism by adding a combination inducer, which induces each of the organoids to be combined, to the mixed culture medium, after the mixing of the culture media, and then incubating the mixed culture medium.

According to a further embodiment of the present invention, in the method of forming the mini-brain structure according to the present invention, each of the organoids present in respectively different regions in the brain may be formed by inducing differentiation of an induced pluripotent stem cell using a specific culture medium, and may be cultured while being suspended in the specific culture medium.

According to a further embodiment of the present invention, in the method of forming the mini-brain structure according to the present invention, the regulating of the culture medium composition may be performed in a manner of gradually mixing one culture medium in which one organoid is suspended with another culture media in which an equal amount of another organoid is suspended over a predetermined time.

According to a further embodiment of the present invention, in the method of forming the mini-brain structure according to the present invention, in the mixing of the culture media, the container for placing the organoids may be used to enable each of the organoids to be adjacent in the mixed culture medium and to control an adjacent position of each of the organoids.

According to a further embodiment of the present invention, in the method of forming the mini-brain structure according to the present invention, the combination inducer may include one or more selected from the group consisting of Matrigel, collagen, gelatin, and a brain extracellular matrix extracted from an animal brain tissue.

### Advantageous Effects

The present invention can obtain the following effects by the above-mentioned embodiments.

In the present invention, organoids present in respectively different regions in the brain, such as the cerebrum, midbrain, cerebellum, and thalamus, are formed by using induced pluripotent stem cells and combined into a single organism. As a result, actual brain functions can be further effectively realized.

In addition, in the present invention, the organoids present in the respectively different regions in the brain are selectively combined into a single organism depending on the purpose of using the brain organoids. As a result, the purpose of using the brain organoids can be achieved while improving convenience and cost-effectiveness.

### DESCRIPTION OF DRAWINGS

FIG. 1 is a reference diagram illustrating a manufacturing process of a mini-brain structure according to an embodiment of the present invention;

FIG. 2 is a diagram showing confocal microscope images for confirming the differentiation status of brain region-specific organoids used for the formation of a mini-brain structure according to an embodiment of the present invention;

FIG. 3 is a diagram showing the measurement results of electrical signals for brain region-specific organoids used for the formation of a mini-brain structure according to an embodiment of the present invention, the measurement performed using MEA;

FIGS. 4 and 5 are diagrams illustrating a manufacturing process of a mini-brain structure according to an embodiment of the present invention;

FIG. 6 is a diagram showing a microscopic image for illustrating a manufacturing process of a mini-brain structure according to an embodiment of the present invention; and

FIG. 7 is a diagram showing microscopic images for illustrating a manufacturing process of a mini-brain structure according to another embodiment of the present invention.

### EXPLANATION OF REFERENCE NUMERAL IN THE DRAWINGS

1: Container for placing organoid

### BEST MODE

Hereinafter, a mini-brain structure and a method of forming the mini-brain structure according to the present invention will be described with reference to the accompanying drawings. Unless otherwise defined, all terms including technical and scientific terms used herein have the same meaning as commonly understood by those skilled in the art to which this invention belongs. When terms used herein discord from the commonly understood meaning, the terms will be interpreted as defined herein. In the following description, it is to be noted that, when the functions of conventional elements and the detailed description of elements related with the present invention may make the gist of the present invention unclear, a detailed description of those elements will be omitted. Unless the context clearly indicates otherwise, it will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof.

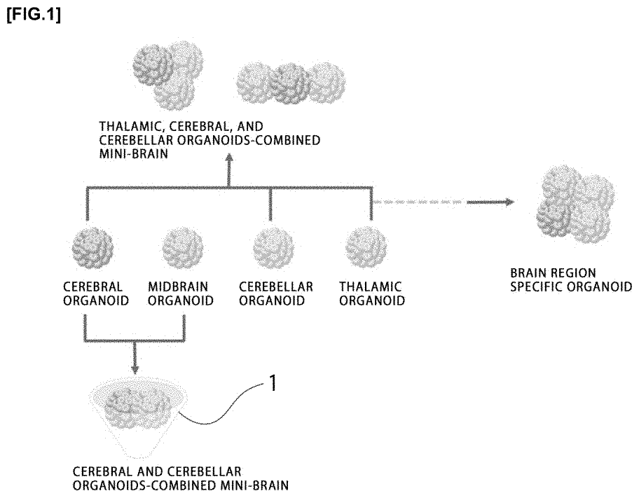

Referring to FIGS. 1 to 7, a mini-brain structure, according to an embodiment of the present invention, includes a plurality of brain organoids. The brain organoids of the plurality of brain organoids are present in respectively different regions in the brain, and the plurality of brain organoids are combined into a single organism.

The brain organoid refers to a three-dimensional cell aggregate similar to brain tissue, that is, differentiated into a brain-like tissue using induced pluripotent stem cells. The brain organoid is composed of tissue similar to a specific brain region (for example, the cerebrum, midbrain, cerebellum, thalamus, and the like). For example, an organoid containing tissue similar to the cerebrum, a specific brain region, is referred to as a cerebral organoid, an organoid containing tissue similar to the midbrain, a specific brain region, is referred to as a midbrain organoid, an organoid containing tissue similar to the cerebellum, a specific brain region, is referred to as a cerebellar organoid, and an organoid containing tissue similar to the thalamus, a specific brain region, is referred to as a thalamic organoid. Existing brain organoids are used for identification of the causes of diseases and confirmation of drug efficacy. However, it is problematic to realize complex human brain functions, which are organically combined, only with existing specific brain organoids based on limited regions. Thus, the purpose of using the brain organoids is difficult to be achieved. In the present invention, the brain region-specific organoids are combined into a single organism to enable the further effective realization of actual brain functions.

In the formation of the mini-brain structure in the present invention, as illustrated in FIG. 1, some organoids are selected from the plurality of brain organoids depending on the purpose of using the mini-brain structure and combined to form the single organism. The influence of all types of diseases, drugs, and the like on each brain region is not necessarily the same. In addition, depending on the types of diseases, drugs, and the like, affected brain regions may vary. For example, assuming that specific drug A affects the thalamus, cerebrum, and cerebellum while specific drug B affects the thalamus and cerebellum, a mini-brain structure formed by combining thalamic, cerebral, and cerebellar organoids is preferably used when using the mini-brain structure to confirm the effect of drug A. In addition, a mini-brain structure formed by combining thalamic and cerebellar organoids is preferably used when using the mini-brain structure to confirm the effect of drug B. That is, the mini-brain structure is formed by selectively combining the organoids present in respectively different regions in the brain into the single organism depending on the purpose of using the mini-brain structure. As a result, the purpose of using the brain organoids can be achieved while improving convenience and cost-effectiveness.

In addition, in the formation of the mini-brain structure according to the present invention, as shown in FIG. 1, the mini-brain structure may have a shape that varies depending on a combining position of the organoids present in respectively different regions in the brain. For example, in the mini-brain structure formed by combining the thalamic, cerebral, and cerebellar organoids, when the thalamic, cerebral, and cerebellar organoids are combined in a row, signals generated from the thalamic organoids due to stimulation affect the cerebral organoids, thereby generating signals therein. In addition, the signals generated from the cerebral organoids affect the cerebellar organoids, so a process of generating signals can be confirmed. In addition, when the thalamic, cerebral, and cerebellar organoids are combined in an aggregate form, signals generated from the thalamus due to stimulation simultaneously affect the cerebral and cerebellar organoids. As a result, processes of generating signals in the cerebral and cerebellar organoids can be confirmed. That is, according to the purpose of using the mini-brain structure, the mini-brain structure having shapes that vary depending on the combining position of the organoids may be formed, thereby effectively confirming the interaction between the brain region-specific organoids.

Referring to FIGS. 1 to 7, a method of forming a mini-brain structure according to another embodiment of the present invention includes: selecting organoids present in one or more specific brain regions depending on a purpose of using a mini-brain structure; regulating a culture medium composition by homogenizing each of the culture media in which the respective selected organoids are suspended; mixing the homogenized culture media in one container to produce a mixed culture medium after the regulating of the culture medium composition so that the organoids become close to each other; inducing each of the organoids to be combined into a single organism by adding a combination inducer, which induces each of the organoids to be combined, to the mixed culture medium, after the mixing of the culture media, and then incubating the mixed culture medium; and the like.

In the selecting of the organoids, the organoids present in respectively different regions in the brain are selected depending on the purpose of using the mini-brain structure. As described above, depending on the types of diseases, drugs, and the like, affected brain regions may vary. Thus, in the selecting, the organoids present in the respectively different regions in the brain are selected depending on the purpose of using the mini-brain structure. For example, assuming that specific drug A affects the thalamus, cerebrum, and cerebellum, in the selecting of the organoids, the thalamic, cerebral, and cerebellar organoids are selected.

In the regulating of the culture medium composition, each of the culture media in which the respective selected organoids are suspended is homogenized. The brain region-specific organoids are formed by inducing the differentiation of the induced pluripotent stem cells with the use of a specific medium, and are cultured while being suspended in the specific medium. As a result, when mixing each of the homogenized culture media in which the respective selected organoids are suspended as they are, a problem of organoid death may occur. As a result, in the regulating of the culture medium composition, one culture medium in which one organoid is suspended is gradually mixed with another culture media in which an equal amount of another organoid is suspended over a predetermined time. That is, the composition of the homogenized culture medium in which the respective selected organoids are cultured is the same.

In the mixing of the culture media, the homogenized culture media are mixed in one container, after the regulating of the culture medium composition so that the organoids become close to each other. For example, in the mixing of the culture media, the respective organoids are allowed to be close to each other by mixing the homogenized culture media in which the respective selective organoids are suspended in a culture dish. In addition, as illustrated in FIG. 1, a container 1 for placing the organoids, such as a conical tube, may be used to facilitate the adjacency of each of the organoids in the mixed culture medium and to control the adjacent position of each of the organoids.

In the inducing of the organoid combination, each of the organoids are combined into a single organism by adding a combination inducer, which induces each of the organoids to be combined, to the mixed media, after the mixing of the culture media, and then culturing the same. The combination inducer may include one or more selected from the group consisting of Matrigel, collagen, gelatin, and a brain extracellular substrate extracted from animal brain tissue.

Hereinafter, the present invention will be described in more detail with reference to embodiments. However, the embodiments disclosed herein are only for illustrative purposes of the embodiments of the present invention, and the spirit and scope of the present invention are not limited thereto.

### <Example 1> Formation of Specific Brain-Region Organoids

1. Formation of 3D Spheroid Using Human Induced Pluripotent Stem Cells

In a culture vessel coated with laminin-based iMatrix-511, human induced pluripotent stem cells (hiPSCs) were cultured using a StemFit Basic002 medium containing 10 μM ROCK inhibitor Y27632 and bFGF. When the cultured hiPSCs accounted for 70% to 80% of the culture vessel, the hiPSCs were separated into single cells by being treated with TrypLE Select. Then, the separated single cells were resuspended after centrifugation and dispensed in an ultra-low attachment 96 well plate at 100,000 cells/well to form spheroids. Hereinafter, specific brain-region organoids were each independently formed using the spheroids.

2. Formation of Cerebral Organoids

(1) Neural Differentiation Induction

A neural induction medium containing a 1× N2 supplement, a 1× GlutaMAX supplement, 1× MEM-NEAA, and 1 μg/ml of heparin was prepared in DMEM-F12. Then, the spheroids were cultured for 4 to 5 days using the neural induction medium.

(2) Cerebral Organoid Patterning

Thereafter, the spheroids on which a germ layer was formed were embedded with Matrigel, and then cultured for 5 days using a cerebral organoid differentiation medium prepared by mixing a 0.5× N2 supplement, 62.5 μl of insulin, 1× GlutaMAX supplement, 0.5× MEM-NEAA, 1× penicillin-streptomycin, 10 μM 2-mercaptoethanol, and a 1× B27 supplement without vitamin A in a medium in which DMEM-F12 and a neurobasal medium were mixed in a ratio of 1:1.

(3) Cerebral Tissue Growth and Final Differentiation

Next, once neuroepithelial buds were formed, the cerebral organoid differentiation medium was replaced with a 1× B27 supplement containing vitamin A to induce differentiation for about 15 days. As a result, cerebral organoids suspended in the medium (final medium) were formed.

2. Formation of Midbrain Organoids

(1) Neural Differentiation Induction

The spheroids were cultured for 48 hours using a neuronal induction medium prepared by mixing a 1× N2 supplement, 1× B27 without vitamin A, 1% Glutamax, 1% MEM-NEAA, 0.175 μL of 2-mercaptoethanol, 1 μg/mL of Heparin, 10 μM SB431542, 200 ng/mL of Noggin, 0.8 μM CHIR99021, and a 10 μM ROCK inhibitor in a medium in which DMEM-F12 and a neurobasal medium containing Antibiotic-Antimycotic were mixed in a ratio of 1:1. Then differentiation was induced for 48 hours using the medium in which the ROCK inhibitor was removed.

(2) Midbrain Organoid Patterning

Thereafter, the resulting product of the neural differentiation induction was cultured for 48 hours using a midbrain patterning medium prepared by mixing a 1× N2 supplement, 1× B27 without vitamin A, 1% Glutamax, 1% MEM-NEAA, 0.175 μL of 2-mercaptoethanol, 1 μg/mL of Heparin, 10 μM SB431542, 200 ng/mL of Noggin, 0.8 μM CHIR99021, 100 ng/mL (or 200 ng/mL) of SHH, and 100 ng/mL of FGF8 in a medium in which DMEM-F12 and a neurobasal medium containing Antibiotic-Antimycotic were mixed in a ratio of 1:1.

(3) Midbrain Tissue Growth

Next, the patterned organoids were embedded with Matrigel, and cultured for 24 hours using a tissue growth induction medium prepared by mixing a 1× N2 supplement, 1× B27 without vitamin A, 1% Glutamax, 1% MEM-NEAA, 0.175 μL of 2-mercaptoethanol, 2.5 μg/mL of insulin, 200 ng/mL of laminin, 100 ng/mL (or 200 ng/mL) of SHH, 100 ng/mL of FGF8, and penicillin/streptomycin in the neurobasal medium.

(4) Final Differentiation

Thereafter, the resulting product of the midbrain tissue growth was shaken at 70 rpm in a final differentiation medium prepared by mixing a 1× N2 supplement, 1× B27 without vitamin A, 1% Glutamax, 1% MEM-NEAA, 0.175 μL of 2-mercaptoethanol, 10 ng/mL of BDNF, 10 ng/mL of GDNF, 100 μM of ascorbic acid, 125 μM db-cAMP, and penicillin/streptomycin in the neurobasal medium to induce final differentiation. As a result, midbrain organoids suspended in the medium (final medium) were formed.

4. Formation of Cerebellar Organoids

(1) Basal Medium Preparation

A 1× N2 supplement, a 1× B27 supplement, 5 μg/mL of insulin, 1.5 mM L-glutamine, 100 μM non-essential amino acids (NEAA), 100 U/L of Pen/Strep, and 10 μM beta-mercaptoethanol were mixed in a medium in which DMEM-F12 and a Neurobasal medium were mixed in a ratio of 1:1 to prepare a neural maintenance medium (NMM), which is a basal medium for the formation of cerebellar organoids.

(2) Neural Differentiation Induction

The spheroids were cultured for 3 days in a medium prepared by mixing 4 ng/ml of FGF2 and a 10 μM ROCK inhibitor in the neural maintenance medium (NMM), and then cultured for 4 days in the medium in which the 10 μM ROCK inhibitor was removed.

(3) Cerebellar Organoid Patterning

Thereafter, the resulting product of the neural differentiation induction was cultured for 7 days using a medium prepared by mixing 4 ng/ml of FGF2, 1 μM retinoic acid, and 100 ng/ml of FGF8B in the NMM.

(4) Cerebellar Tissue Growth

Next, the patterned organoids were cultured for 3 days using a medium prepared by mixing 4 ng/ml of FGF2, 100 ng/ml of FGF4, and 100 ng/ml of FGF8B in the NMM, cultured for 5 days in the medium in which FGF2 and FGF4 were removed, and cultured for 7 days in a medium prepared by mixing 100 ng/ml of BDNF and ng/ml of GDNF in the NMM.

(5) Final Differentiation

Thereafter, the resulting product of the cerebellar tissue growth was cultured for about 7 days by adding 3 ng/ml of SAG, 100 ng/ml of NT3, and 25 mM KCl to the medium. As a result, cerebellar organoids suspended in the medium (final medium) were formed.

5. Formation of Thalamic Organoids

(1) Neural Differentiation Induction

Differentiation was induced using induction medium prepared by mixing 15% KSR, 1% MEM-NEAA, 1% Glutamax, 100 mM b-Mercaptoethanol, 100 nM LDN-193189, 10 mM SB-431542, 4 mg/ml of insulin, 5% heat-inactivated FBS, and 50 mM Y27632 in DMEM-F12. Then, the spheroids were cultured by replacing the medium with the medium in which FBS was removed after 2 days and replacing the medium with the medium in which Y27632 was removed after 4 days and 6 days.

(2) Thalamic Organoid Patterning

The resulting product of the neural differentiation induction was cultured for 8 days using a patterning media prepared by mixing 0.15% Dextrose, 100 mM 2-mercaptoethanol, a 1× N2 supplement, a 1× B27 supplement minus vitamin A, 30 ng/ml of BMP7, and 1 mM PD325901 in DMEM-F12 while being shaken at 80 rpm.

(3) Thalamic Tissue Growth and Final Differentiation

The patterned thalamic organoids were cultured for about 10 days using a final differentiation medium prepared by mixing a 0.5× N2 supplement, 0.5× B27, 1% Glutamax, 0.5% MEM-NEAA, 0.025% Insulin, 50 mM 2-mercaptoethanol, 20 ng/ml of BDNF, and 200 nM ascorbic acid in a medium in which DMEM-F12 and a neurobasal medium containing Antibiotic-Antimycotic were mixed in a ratio of 1:1. As a result, thalamic organoids suspended in the medium (final medium) were formed.

### <Example 2> Brain Region-Specific Organoids were Confirmed to be Formed

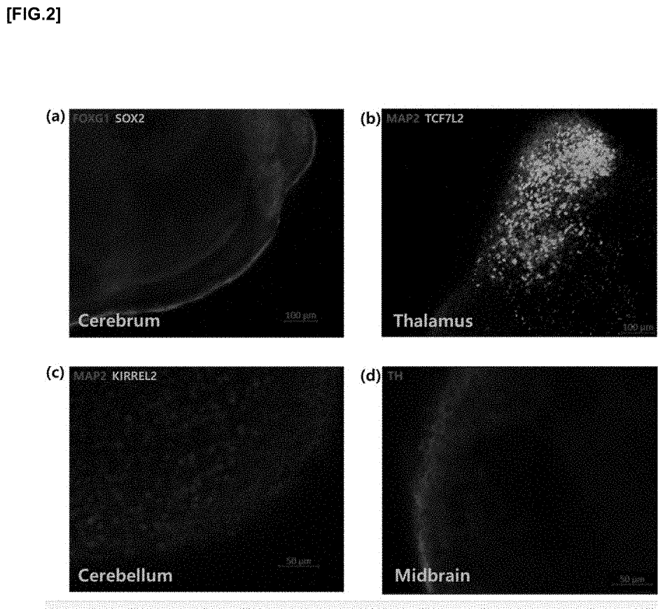

1. Confirmation Using Specific Marker for Specific Brain Region.
(1) Immunostaining was performed to confirm whether specific markers for specific brain regions were expressed in each of the organoids formed in Example 1. Specifically, the cerebral organoids were stained with FOXG1 and SOX2, the thalamic organoids were stained with TCF7L2 and MAP2, the cerebellar organoids were stained with KIRREL2 and MAP2, and the midbrain organoids were stained with TH to examine fluorescence with a confocal microscopy. The results thereof are shown in FIG. 2.
(2) Referring to FIG. 2, it was confirmed that cerebrum-specific proteins were expressed in the cerebral organoids, thalamus-specific proteins were expressed in the thalamic organoids, cerebellum-specific proteins were expressed in the cerebellar organoids, and midbrain-specific proteins were expressed in the midbrain organoids.

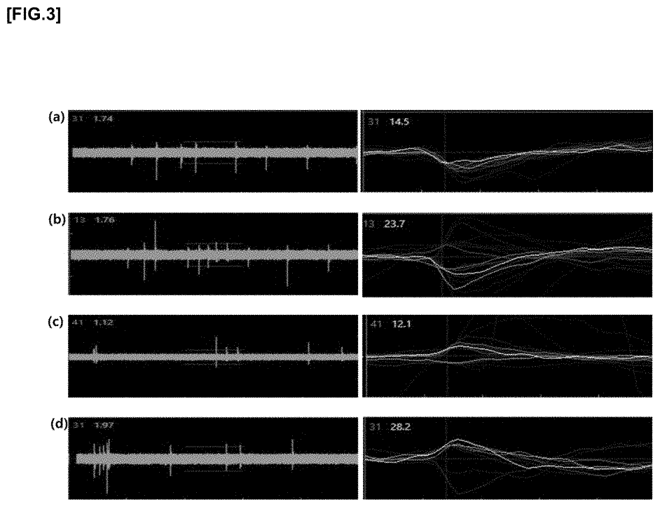

2. Confirmation Using Bioelectrical Signals Generated Using MEA
(1) Multi-electrode array (MEA) system is a device that measures the action potential of neurons. Changes in the action potential of brain organoids were measured so as to be used for the analysis of electrophysiological variation, changes in neuron ion channel, signal transmission speed, and analysis of signal transmission detection between neurons. Accordingly, the bioelectrical signals of each of the organoids formed in Example 1 was examined using MEA. Specifically, a MEA substrate was coated with polyetherimide (PEI) and then re-coated with laminin to place each of the organoids formed in Example 1 thereon and measure the signals. The results thereof are shown in FIG. 3 (FIG. 3A shows the Experimental results of the cerebral organoids, FIG. 3B shows experimental results of the thalamic organoids, FIG. 3C shows experimental results of the cerebellar organoids, and FIG. 3D shows experimental results of the midbrain organoids).
(2) Referring to FIG. 3, it is confirmed that each of the organoids generates different spontaneous electrical signals, indicating that each of the organoids is composed of different neurons.

### <Example 3> Formation of Mini-Brain Structures

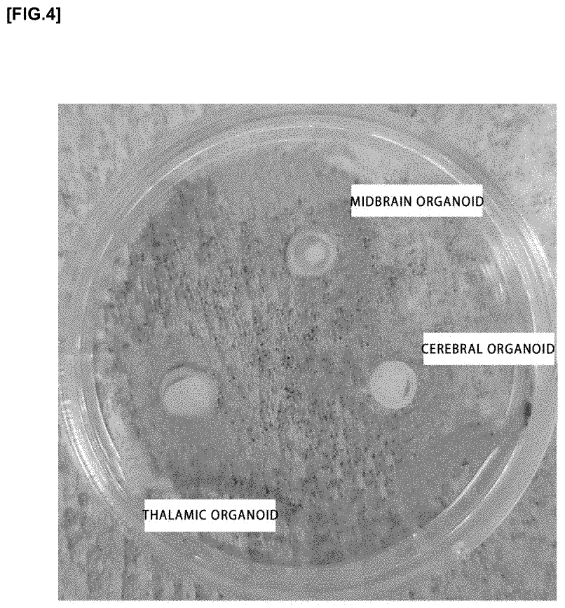

1. Formation of Mini-Brain Structure 1
(1) To form a mini-brain structure 1 by combining thalamic, cerebral, and midbrain organoids cultured while being suspended in a culture medium, a culture medium composition of each of the organoids formed in Example 1 was homogenized. Specifically, a final culture medium in which the thalamic organoids were suspended was gradually combined with final culture media for the cerebral and midbrain organoids in an equal amount over a week. In addition, a final culture medium in which the cerebral organoids were suspended was gradually combined with final culture media for the thalamic and midbrain organoids in an equal amount over a week. Furthermore, a final culture medium in which the midbrain organoids were suspended was gradually combined with final culture media for the thalamic and cerebral organoids in an equal amount over a week. In the culture medium in which each of the organoids was cultured, a concentration ratio of the final culture mediums for the thalamic, cerebral, and midbrain organoids were 1:1:1. That is, each of the culture media in which each of the organoids was cultured had the same composition.
(2) Then, as shown in FIG. 4, the culture media in which the respective organoids were suspended were mixed, and the thalamic, cerebral, and midbrain organoids were allowed to be close to each other.
(3) Next, Matrigel was added to the mixed culture media to induce each of the organoids to be combined, and the mixed culture media were incubated for 2 days to form the mini-brain structure 1.

2. Formation of Mini-Brain Structure 2
(1) To form a mini-brain structure 2 by combining cerebellar, cerebral, and midbrain organoids cultured while being suspended in a culture medium, a culture medium composition of each of the organoids formed in Example 1 was homogenized. Specifically, a final culture medium in which the cerebellar organoids were suspended was gradually combined with final culture media for the cerebral and midbrain organoids in an equal amount over a week. In addition, a final culture medium in which the cerebral organoids were suspended was gradually combined with final culture media for the cerebellar and midbrain organoids in an equal amount over a week. Furthermore, a final culture medium in which the midbrain organoids were suspended was gradually combined with final culture media for the cerebellar and cerebral organoids in an equal amount over a week. In the culture medium in which each of the organoids was cultured, a concentration ratio of the final culture mediums for the cerebellar, cerebral, and midbrain organoids were 1:1:1. That is, each of the culture media in which each of the organoids was cultured had the same composition.
(2) Then, the culture media in which the respective organoids were suspended were mixed, and the cerebellar, cerebral, and midbrain organoids were allowed to be close to each other.
(3) Next, Matrigel was added to the mixed culture media to induce each of the organoids to be combined, and the mixed culture media were incubated for 2 days to form the mini-brain structure 2.

### <Example 4> Confirmation that the Mini-Brain Structure was Fabricated

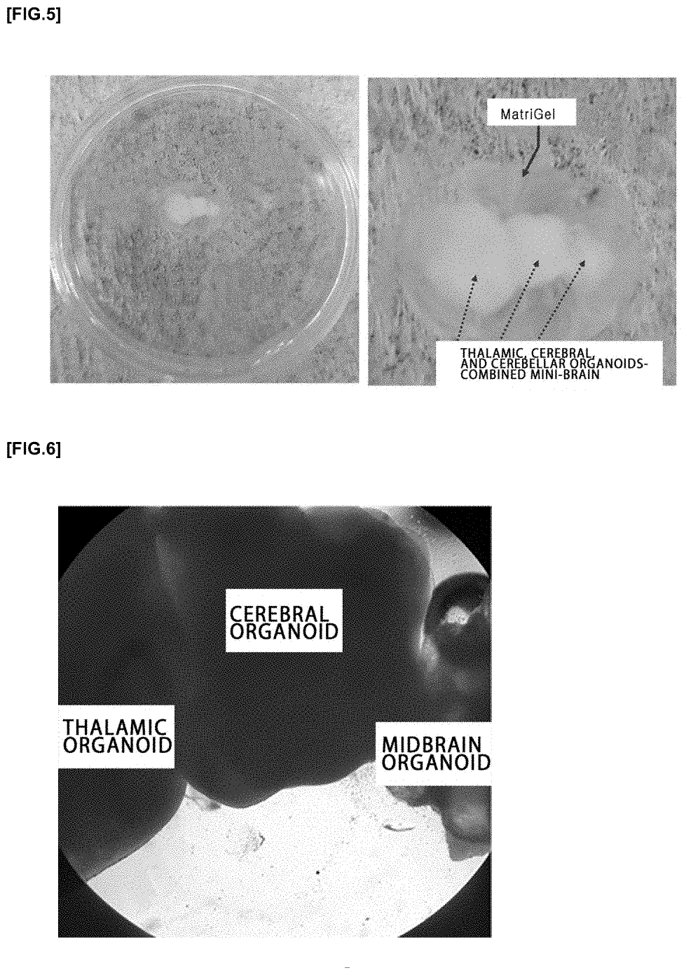

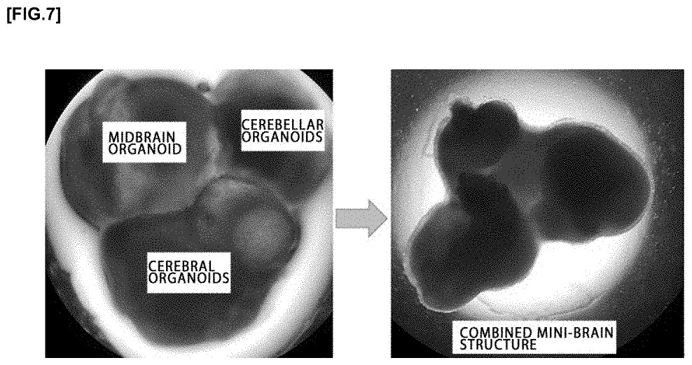

1. The mini-brain structure 1 formed in Example 3-1 was photographed with a digital camera, and the results thereof were shown in FIG. 5. In addition, the mini-brain structure 1 was examined with a microscope in a process of combining each of the organoids, and the result thereof was shown in FIG. 6. Furthermore, the mini-brain structure 2 formed in Example 3-2 was independently examined with a microscope in a process of combining each of the organoids and after the combining process was completed. The results thereof were shown in FIG. 7.
2. Referring to FIGS. 5 and 6, it is confirmed that in the mini-brain structure 1, the thalamic, cerebral, and midbrain organoids are combined in a row, thereby forming a single organism. Referring to FIG. 7, it is confirmed that the midbrain, cerebellar, and cerebral organoids are connected to each other, thereby forming a single organism.

Although the applicant has described the preferred embodiments of the present invention, these embodiments are only one embodiment for implementing the technical spirit of the present invention. In addition, it should be understood that the present invention includes various changes or modifications without departing from the scope and spirit of the invention as disclosed in the accompanying claims.

1. A mini-brain structure comprising a plurality of brain organoids,
wherein the brain organoids of the plurality of brain organoids are present in respectively different regions in the brain, and
the plurality of brain organoids is combined into a single organism.

2. The mini-brain structure of claim 1, wherein the mini-brain structure is formed by selecting some organoids from the plurality of brain organoids and combining the selected organoids depending on a purpose of using the mini-brain structure.

3. The mini-brain structure of claim 1, wherein the mini-brain structure has a shape that varies depending on a combining position of the organoids according to a purpose of using the mini-brain structure.

4. A method of forming a mini-brain structure, the method comprising:
selecting organoids present in one or more specific brain regions depending on a purpose of using a mini-brain structure;
regulating a culture medium composition by homogenizing each of culture media in which the respective selective organoids are suspended; and
mixing the homogenized culture media in one container to produce a mixed culture medium after the regulating of the culture medium composition so that the organoids become close to each other.

5. The method of claim 4, further comprising inducing each of the organoids to be combined into a single organism by adding a combination inducer, which induces each of the organoids to be combined, to the mixed culture medium, after the mixing of the culture media, and then incubating the mixed culture medium.

6. The method of claim 5, wherein each of the organoids present in one or more specific brain regions is formed by inducing differentiation of an induced pluripotent stem cell using a specific culture medium, and is cultured while being suspended in the specific culture medium.

7. The method of claim 6, the regulating of the culture medium composition is performed in a manner of gradually mixing one culture medium in which one organoid is suspended with another culture media in which an equal amount of another organoid is suspended over a predetermined time.

8. The method of claim 5, wherein in the mixing of the culture media, the container for placing the organoids is used to enable each of the organoids to be adjacent in the mixed culture medium and to control an adjacent position of each of the organoids.

9. The method of claim 5, wherein the combination inducer comprises one or more selected from the group consisting of Matrigel, collagen, gelatin, and a brain extracellular matrix extracted from an animal brain tissue.
